(19) United States
(12) Patent Application Publication
(10) Pub. No.: US 2010/0184197 A1
(43) Pub. Date: Jul. 22, 2010

(54) Methods For Harvesting Biological Materials Using Membrane Filters

(76) Inventors: Longying Dong, Elmira, NY (US); Kenneth Joseph Drury, Big Flats, NY (US); Andrei Gennadyevich Fadeev, Elmira, NY (US)

Correspondence Address: CORNING INCORPORATED, SP-TI-3-1, CORNING, NY, 14831, US

(21) Appl. No.: 12/357,901

(22) Filed: Jan. 22, 2009

(51) Int. Cl. C12N 1/12

(52) U.S. Cl. 435/257.1; 435/261

(57) ABSTRACT

The present disclosure relates to methods for harvesting biological materials, such as, for example, microalgal cells, using membrane filters, such as ceramic-based membrane filters.

### FIELD OF THE DISCLOSURE

The present disclosure relates to methods for harvesting biological materials, including methods for harvesting microalgal cells for biodiesel production and food or nutritional supplements, using membrane filters.

### BACKGROUND

Increasingly high oil prices, limited fossil fuel reserves, and environmental concerns about global warming have driven a tremendous interest in developing alternative, renewable, and sustainable energy over the last decade. Plants possess a natural system to convert solar energy to renewable and storable chemical energy via photosynthesis, and therefore, high oil plants, such as rapeseed, palms, soybeans and corn, have been used as raw materials for biofuel production. In contrast to biofuels from food crops or cellulosic materials, microalgae, organisms capable of photosynthesis that are less than 0.4 mm in diameter, including diatoms and cyanobacteria, may be a more attractive source for oil production due, in part, to its less complex structure, fast growth rate, high oil content, lack of competition with the food supply, and capability for growing on land not suitable for agriculture, such as near by a power plant.

Commonly used technologies for harvesting microalgae biomass include centrifugation, flocculation, and filtration. However, these techniques as used in the art may have disadvantages, including, but not limited to, being tedious and/or expensive, such that they are inoperable or impractical on an industrial scale or for a range of biological material sizes, and may further cause sample damage and/or contamination. Nonetheless, as global energy demands grow, the need for renewable and sustainable biofuels has become urgent, and thus simple and effective methods for harvesting biological materials are desired.

Microalgae-based biofuels, however, are not yet being made on a commercial scale due to engineering and process challenges, and microalgae-based fuels are more costly than petroleum-based fuels. For example, one of the technical hurdles for biodiesel production is microalgae harvesting on a large scale. According to the estimation in Gudin C. & Therpenier C., Bioconversion of Solar Energy into Organic Chemicals by Microalgae, Adv. Biotechnol Processes (1986) 6:73-110, the cost of harvesting microalgae accounts for 20-30% of total cost of biodiesel production. The difficulties are due, in part, to the combination of the small size of the microalgae (3-30 μm) and their low concentration in the culture medium (typically less than 500 mg/L).

The inventors have now discovered effective methods for harvesting biological materials, including microalgal cells, using membrane filters.

### SUMMARY

In accordance with the detailed description and various exemplary embodiments described herein, the present disclosure relates to methods for harvesting biological materials, such as, for example microalgal cells, using membrane filters. In various exemplary methods, biological materials are harvested by passing a sample comprising the biological materials through a membrane filter, wherein the filter support comprises a porous material. In additional exemplary embodiments, the sample containing the biological materials may be circulated through the membrane filter.

### BRIEF DESCRIPTION OF THE DRAWINGS

The accompanying drawings are included to provide a further understanding of the invention, and are incorporated in and constitute a part of this specification. The drawings are not intended to be restrictive of the invention as claimed, but rather are provided to illustrate exemplary embodiments of the invention and, together with the description, serve to explain the principles of the invention.

FIG. 1 is a schematic diagram of an exemplary membrane filter, according to one embodiment of the invention.

FIG. 2 is a schematic diagram of an exemplary apparatus for harvesting biological material, according to one embodiment of the invention.

FIG. 3 is a graphical representation of performance results from the exemplary methods described in Example 1 herein.

FIG. 4 is a graphical representation of the impact of initial biomass density on filtration flux as described in Example 2 herein.

FIG. 5 is a graphical representation of the impact of pump flow rate on filtration flux as described in Example 2 herein.

### DETAILED DESCRIPTION

It is to be understood that both the foregoing general description and the following detailed description are exemplary and explanatory only and are not restrictive of the invention, as claimed. Other embodiments of the invention will be apparent to those skilled in the art from consideration of the specification and practice of the invention disclosed herein. It is intended that the specification and examples be considered as exemplary only, with the true scope and spirit of the invention being indicated by the claims.

The present disclosure relates to methods for harvesting biological materials, including microalgae, using membrane filters. In various embodiments, the methods may comprise passing a sample comprising at least one biological material, such as a diluted biological suspension, through at least one membrane filter. In at least one exemplary embodiment, the sample comprising at least one biological material is passed or circulated through the at least one membrane filter one or more times, such as two or more times. The presently disclosed methods may simplify the processes currently in use for harvesting biological materials, such as at the industrial scale, including, but not limited to, microalgae harvesting for biofuel production.

As used herein, the terms “harvest,” “harvesting,” and variations thereof, mean increasing the biomass density or concentration of biological materials obtained by various methods described herein. Harvesting of biological material may be evidenced by, for example, any reduction in fluid volume and/or increase in biomass density. As used herein, the phrases “reduced fluid volume,” “increased biomass density,” and variations thereof mean any reduction in fluid volume or increase in biomass density relative to that of the original sample. By way of example only, the harvesting methods may reduce the fluid volume of a sample by at least 20 percent, such as at least 40, 60, 80, 90 or even 95 percent or more relative to the original sample. In various exemplary embodiments, the harvesting may reduce the fluid volume until a slurry or paste containing the biological material is obtained.

The industrial biomass density of microalgae cell suspension may vary depending on the culture conditions, including light intensity, temperature, and CO₂ and nutrient supplies. Typically, however, biomass density produced from a controlled photobioreactor is higher than that from an open pond culture. Thus, as non-limiting examples, the biomass density obtained under different culture conditions may vary from less than 10 mg/L to greater than 500 mg/L, for example from less than 500 mg/L to greater than 1500 mg/L. The presently disclosed methods are capable of harvesting very low density biomasses, for example less than 10 mg/L; on the other hand the methods are also capable of harvesting very high density biomasses, for example greater than 5,000 mg/L.

As used herein, the terms “biological material,” “biological materials,” “biomass,” and variations thereof, are intended to include plant and animal matter, for example, but not limited to, microalgae and bacteria cells. The terms “diluted biological suspension,” “biological suspension,” “suspension,” and variations thereof mean a suspension of biological materials in a liquid or slurry. Non-limiting examples of suspensions include, inter alia, microalgae suspended in culture medium, which may be a pH adjusted fluid containing cell nutrients, such as those obtained from open ponds or from enclosed photobioreactors. Additional examples include oils and organic solvents containing biological materials, for example, aqueous microalgae suspensions containing oils or lipids.

As used herein, the terms “membrane filter,” “filter,” and variations thereof, are intended to include porous monolithic bodies or supports optionally coated with at least one membrane layer. The monolithic body or support may be formed from any suitable porous material, including, for example, ceramic materials and carbon-based materials. Ceramic materials include, but are not limited to, those comprising mullite, cordierite, alumina, and silicon carbide. Carbon-based materials include, but are not limited to, synthetic carbon-containing polymeric material (which may be cured or uncured); activated carbon powder; charcoal powder; coal tar pitch; petroleum pitch; wood flour; cellulose and derivatives thereof; natural organic materials, such as wheat flour, wood flour, corn flour, nut-shell flour; starch; coke; coal; or mixtures thereof. In some embodiments, the carbon-based material comprises a phenolic resin or a resin based on furfuryl alcohol. In some embodiments, the carbon-based material is a carbonized and optionally activated form of the materials mentioned above.

In at least one exemplary embodiment, the monolithic body is comprised of a porous ceramic material. As a non-limiting example, the porous monolithic body may be made from a ceramic composition selected from mullite (3Al₂O₃.2SiO₂), alumina (Al₂O₃), silica (SiO₂), cordierite (2MgO.2Al₂O₃.5SiO₂), silicon carbide (SiC), titania (TiO₂), alumina-silica mixtures, glasses, inorganic refractory materials, and porous metal oxides.

In at least one embodiment, the monolithic body is comprised of a porous ceramic mullite, such as the mullite compositions disclosed and described in U.S. Pat. Nos. 6,238,618 and 6,254,822, the entire disclosures of which are incorporated by reference herein. In at least some embodiments, mullite may offer significant strength retention, such as in corrosive environments, and also an extended pH operating range. In addition to excellent pH stability, a mullite body may, in at least certain embodiments, have virtually no restriction with respect to the types of organic fluids that may be passed through it. Monoliths comprising mullite can, in at least certain embodiments, be back pulsed as well as steam sterilized. Mullite materials may also have Food and Drug Administration clearance for contact with food. These advantages may be significant for various embodiments and species of microalgae cell harvesting, such as Spirulina cells, which are typically cultured in high pH medium and may be used for food or nutrition supply. The skilled artisan will appreciate, however, that other materials may be more appropriate, for example, for filters intended for other applications.

The porous material that forms the monolith or support is comprised of an interconnected matrix or network of pores which forms a networked plurality of fluid pathways. In various embodiments of the disclosure, the total pore volume or porosity of the monolithic body may range from 20% to 60%, including, for example, 25%, 30%, 35%, 40%, 45%, 50%, 55%, and any range derived therefrom.

In various exemplary embodiments, the pore volume of the monolithic body may consist essentially of pores having pore diameter sizes ranging from 2 μm to 20 μm, including, for example, ranging from 8 μm to 12 μm.

The pore size and total porosity values can be quantified using conventional methods and models known to those of skill in the art. For example, the pore size and porosity can be measured by standardized techniques, such as mercury porosimetry and nitrogen adsorption.

The monolith may further contain “filtrate conduits,” which are channels or conduits arranged to provide a pathway for the filtrate material to flow through the interior of the monolithic body in a stream separate from the retenate or biological suspension. The filtrate conduits may be capable of directing separated filtrate that has permeated the conduit walls to the exterior of the monolithic body for subsequent collection or processing. In various exemplary embodiments, these filtrate conduits may extend from the inlet end to the outlet end of the monolith. The filtrate conduits provide flow paths of lower flow resistance than that of flow through the porous material. In at least one embodiment, the monolith may be constructed such that the filtrate conduits are distributed throughout the body to provide low pressure drop flow paths from the body of porous material to nearby filtrate conduits. Exemplary discrete filtrate conduits are disclosed and described in, for example, U.S. Pat. No. 4,781,831, which is incorporated by reference herein.

In various exemplary embodiments, the filtrate conduits may further comprise one or more channels or slots extending from the conduit to the filtrate collection zone or area. For example, a slot may transversely extend from the longitudinal conduit to the external surface of the monolith. In various embodiments, the channels or slots may be formed at the inlet end or outlet end of the monolith, through the exterior surface of the monolithic body at any point along the length of the conduit, or any combination thereof.

In further embodiments, the filtrate conduits may be plugged or blocked at the inlet end and/or the outlet end by one or more barriers. The barriers may inhibit direct passage of the biological suspension stream into or out of the filtrate conduits at the feed end or the outlet end of the monolith. The barriers may, for example, be plugs of material inserted or introduced into the filtrate conduit. The barriers may optionally be comprised of the same material as the structure, or may be some other suitable material, and the barriers may in at least some embodiments have a porosity similar to or less than that of the structure material. For example, in various exemplary embodiments, the plugs may be comprised of cement, organic sealants, or epoxy.

In at least one exemplary embodiment, the monolithic body does not comprise filtrate conduits. For example, when the monolith has small module diameter, for example less than 50 mm, it may provide adequate filtration without incorporating filtrate conduits. In a further exemplary embodiment, the monolithic body does comprise filtrate conduits. For example, bodies having diameters larger than 50 mm may require filtrate conduits to facilitate the removal of filtrate fluids from the internal portions of the filter body.

In various exemplary embodiments, a bare monolith body may be suitable for harvesting the biological material. For example, for large size microalgae, a monolith body with a suitable pore size distribution (i.e., smaller than the cell diameter) may be used for harvesting the biomass.

In various exemplary embodiments, at least one membrane layer of porous material having smaller pore sizes than the pores of the monolith body may be deposited onto the walls of the fluid passageways in the monolith or support. The membrane layer may be comprised of any suitable porous material, including but not limited to ceramic and carbon-based materials, for example, materials selected from alumina, silica, mullite, glass, zirconia, titania, and combinations thereof. In at least one embodiment, the membrane layer is comprised of alumina, zirconia, silica or titania. In various embodiments, the desired filtration pore size of the membrane filter may be controlled by using a particular coating on fluid passageways. The membrane layer may be applied by conventionally known wet chemistry methods, such as a conventional slip casting process or any other method known to those of skill in the art. In various exemplary embodiments, at least one membrane layer is deposited such that it exhibits a layer thickness ranging of from 5 μm to 150 μm. In additional exemplary embodiments, the pore volume of the membrane layer may comprise pore sizes ranging from 10 nm to 500 nm, for example from 200 nm to 450 nm, and from 200 nm to 400 nm. In at least one embodiment, the at least one membrane layer may optionally be combined with at least one second membrane layer having a smaller pore size, for example less than 200 nm.

In additional exemplary embodiments, an optional membrane film providing a separation function may be applied to the at least one membrane layer or directly to the surfaces of the fluid passageways of the monolithic body or support. In further embodiments, the membrane film providing the separation function may be deposited such that it exhibits a layer thickness ranging from 1 μm to 10 μm, and the membrane film may have a pore size of less than 200 nm.

In at least one exemplary embodiment, the disclosed methods may filter biological material of a range of sizes, for example those of a size ranging from 0.2 μm to 30 μm, such as, for example, ranging from 0.2 μm to 3 μm.

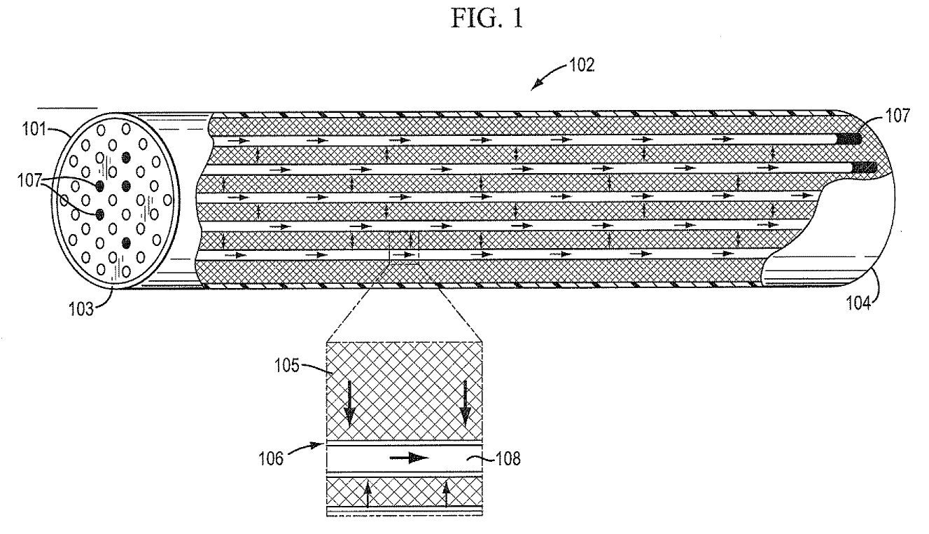

For example, as depicted in FIG. 1, which is a schematic diagram of an exemplary membrane filter, a diluted biological suspension 101 enters the membrane filter 102 at the inlet end 103 and travels through the filter towards the outlet end 104. While traveling through the membrane filter 102, the suspension components of a pre-selected pore size or smaller pass from the porous body 105, through the membrane filter 106, and into the filtrate conduit 108, as depicted by the arrows in FIG. 1. FIG. 1 also depicts that the filtrate conduits 108 may be plugged by barriers 107 at the inlet and/or outlet ends of the monolith.

In various embodiments, the appropriate pore size distribution of the membrane filter may be determined by those skilled in the art based on, for example, the cell size of the biological material and the desired filtration flux. By way of example, the pore size distribution may need to be adjusted based on the diameter of the biological material being filtered in order to maximize filtration flux in certain embodiments. For example, as explained herein, microalgae have thousands of species with sizes ranging from 3 μm to 30 μm. Thus, for example, for a bacteria size blue-green algae, such as for example Synechocystis ps. PCC 6803, the optimal membrane filter pore size may be in the range of 0.2 μm to 1 μm for the desired filtration flux of a particular application. Likewise, for a large size of blue-green algae, such as Spirulina platensis, the optimal membrane filter pore size may be in the range of 1 μm to 3 μm for the desired filtration flux of a particular application.

In various embodiments, the methods described herein may operate at any range of pH. In at least one embodiment, the methods may be performed on samples having pH ranging from 2 to 13, such as 11.

In various exemplary embodiments, the at least one membrane filter may be mounted, for example in at least one housing. The at least one housing may be chosen from any type of housing material, including plastic or metal materials and can be designed in a number of configurations, such as, for example, a 3-A approved sanitary stainless steel design housing or a non-sanitary industrial design housing. In either of those housing types, the membrane filter may optionally be fitted with elastomeric boots that fit over each of the two faces of the filter housing. These boots may be configured to seal the filtrate space, for example to prevent permeate mixing with feed and/or concentrate. One of skill in the art will appreciate that a different type of seal may also be used. Further, in various exemplary embodiments, each membrane filter configuration may be an individual monolith, and may comprise a stainless steel end ring fitting bonded to each end. The appropriate bonding material for any particular application is well within the ability of those skilled in the art to determine. By way of example, in at least one embodiment, the material used to bond the end rings to the monolith may be a polymeric adhesive.

In various exemplary embodiments, passing the biological material through the membrane filter may comprise passing or circulating the biological suspension of various cell sizes and cell densities through at least one membrane filter one or more times, such as, for example, two or more times. In at least one embodiment, two or more membrane filters may be used in series or in parallel. Any method known to those of skill in the art for passing or circulating biological materials through membrane filter(s) may be used. By way of example, a vacuum pump or other similar mechanism may be used as a driving force. The method used and number of times a sample is passed through the membrane filter(s) may easily be determined by those of skill in the art, for example depending on the type of biological materials being harvested and the desired biomass concentration.

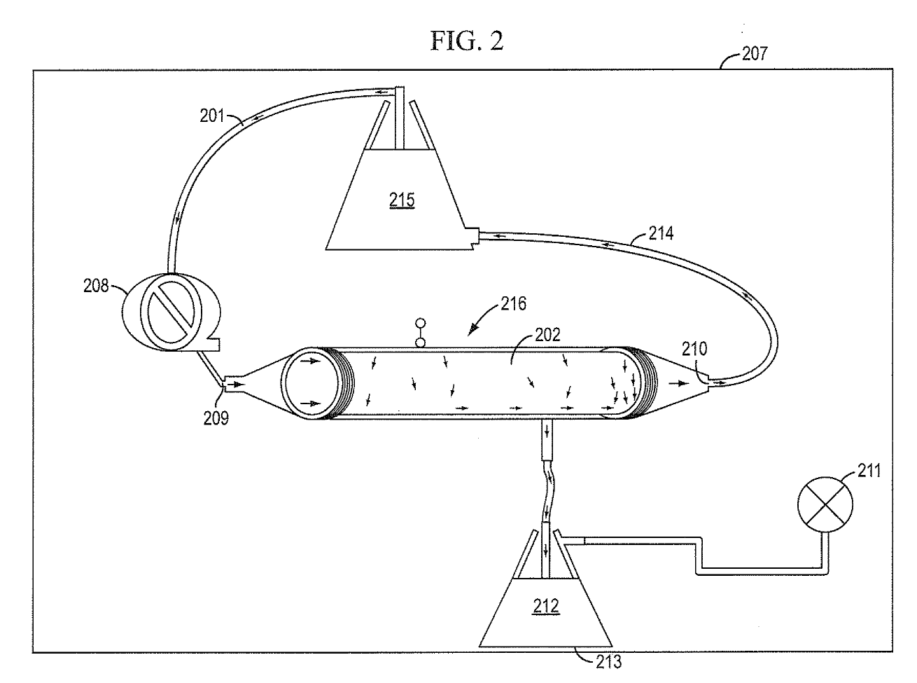

For example, as depicted in FIG. 2, which is a schematic diagram of an exemplary bench-scale apparatus for harvesting biological material, a biological suspension 201 may be continuously circulated through the membrane module 216 using peristaltic pump 208 one or more times. For maximization of volume reduction or biomass concentration, the inlet 209 and outlet 210 of the filter housing may be reduced in diameter from that of the membrane filter 202. A vacuum pump 211 may be used to apply vacuum to the side of the membrane module 216 and provide a driving force for filtration. In various embodiments, the vacuum pump or other similar mechanism may be unnecessary when the circulation is operated at a sufficiently high flow rate to independently produce a driving force, such as, for example, greater than 1000 mL/min, greater than 1200 mL/min, or greater than 1500 mL/min, such as 1595 mL/min. In various exemplary embodiments, the driving force for passing the at least one biological suspension through the at least one membrane filter may range from 0.05 bar to 4 bar. Filtered fluid 212 is continuously collected in a filtrate flask 213, and concentrated cells 214 are returned to the feed container or upper flask 215.

Once the desired volume reduction or density increase of feed stock is obtained by passing or circulating the sample through the at least one membrane filter one or more times, the condensed biological suspension may be recovered. To achieve a higher recovery of the biological material, additional steps may be performed. In various exemplary embodiments, the circulation may be further run at a higher flow rate (i.e., higher than the operating flow rate) prior to recovery for an appropriate length of time as determined by one skilled in the art, such as, for example, 5 minutes, to break down the biological material cake that may form along the walls of the membrane. In additional exemplary embodiments, the pump may be run in reverse to collect the concentrated biological material back to the feed container.

In additional exemplary embodiments of the present disclosure, the filtration system may be regenerated by flushing the system with a fluid, for example, but not limited to, water or culture medium. Flushing the system may recover residual biological materials, such as biological materials trapped or held up in the system tubing and filtrate housing. As a further example, residual biological materials can be substantially fully recovered by flushing the system, thereby achieving nearly 100% recovery. The recovered residual concentrated biological materials may exhibit normal cell viability and may be collected in the feed container or directly pumped into a bioreactor or pond to start a new cycle of biomass production.

In additional exemplary embodiments of the present disclosure, such regeneration of the filtration system by flushing with medium or water may be sufficient to maintain a constant flux, for example to achieve a filtration capacity of 50 g/m² for a 1″×2″ membrane filter without exhibiting a significant drop in flux.

In at least one exemplary embodiment, if a significant drop in flux is observed during filtration, the filter can be cleaned by any method known to those of skill in the art. For example, the filter may be taken out of the system, soaked in commercial bleach, and then rinsed with distilled water. In a further example, the filter may be soaked in commercial bleach for 30 minutes and rinsed with distilled water up to three or more times. In at least one embodiment, this process may substantially fully recover the filtration performance without significant membrane fouling or performance change. By way of example, after using such a bleaching method, a 1″×2″ membrane filter may be capable of concentrating 360 g/m² without membrane fouling and/or performance change. In another embodiment the membrane filter may be cleaned while in place by circulating bleach or other cleaners through the membrane. In addition, the membrane filter may be cleaned by baking it in an oven, for example at a temperature of 500° C.

In at least one exemplary embodiment, the membrane filter may be connected in a loop with an enclosed system, i.e., a bioreactor, or an open pond, and a diluted biological suspension may be circulated through at least one filter one or more times to reduce the volume and concentrate the biomass. In at least one embodiment, the biological suspension may be continuously circulated through at least one filter two or more times, until the desired volume and/or concentration is achieved.

The wet biomass may proceed directly to oil extraction. However, prior to oil extraction, in various exemplary embodiments, the concentrated biomass obtained by the methods described may subsequently be further dewatered, for example by gravity sedimentation, or dried, for example air dried, or may be further concentrated by any other known method.

Unless otherwise indicated, all numbers used in the specification and claims are to be understood as being modified in all instances by the term “about,” whether or not so stated. It should also be understood that the precise numerical values used in the specification and claims form additional embodiments of the invention. Efforts have been made to ensure the accuracy of the numerical values disclosed in the Examples. Any measured numerical value, however, can inherently contain certain errors resulting from the standard deviation found in its respective measuring technique.

As used herein the use of “the,” “a,” or “an” means “at least one,” and should not be limited to “only one” unless explicitly indicated to the contrary. Thus, for example, the use of “the membrane filter” or “a membrane filter” is intended to mean at least one membrane filter.

Other embodiments of the invention will be apparent to those skilled in the art from consideration of the specification and practice of the invention disclosed herein. It is intended that the specification and examples be considered as exemplary only, with a true scope and spirit of the invention being indicated by the claims.

### EXAMPLES

The following examples are not intended to be limiting of the invention as claimed.

### Example 1

Cyanobacteria (Synechocystis sp. PCC 6803), a blue-green algae cell suspension cultured in the modified BG-11 medium (ATCC medium 616) plus 10 mM Hepes buffer (pH 7.4) to maintain the pH, was harvested using a honeycomb mullite-based membrane filter.

The membrane filter used for this experiment was SP-2-1, which is a mullite-based membrane support 2 inches (˜5.08 cm) in length, 1 inch in diameter, and with 56 square fluid passageways (1.85×1.85 mm² each). The total filtration area was 210.5 cm² and the area of cross section or front area is 1.8144 cm².

The ceramic support or monolith had 50% porosity, with a 9 μm mean pore size, and the selective membrane coated on top of the support, which was made of alumina, had a 0.2 μm to 0.4 μm mean pore size.

A bench-scale apparatus similar to that shown in FIG. 2 was used for harvesting Synechocystis ps. PCC 6803 cells from the suspension. The suspension was circulated through the membrane filter using a peristaltic pump. To reduce the dead volume and increase filtration efficiency, the inlet and outlet of the filter housing was reduced to 5/16 inch in diameter from the original 1½ inch diameter of the membrane filter. The feed flow rate of microalgae cell culture suspension through the membrane module was 704 mL/min. Vacuum was applied to the permeate port of the membrane module to provide a driving force of 15 in Hg for filtration.

Upon applying vacuum to the permeate port of the membrane, the culture medium is drawn to a filtrate container (lower flask) through the membrane filter, while the microalgae cells are concentrated in the feed container (upper flask).

The membrane filter performance was evaluated by measuring permeate flux and filtration efficiency. The cell density was measured spectrometrically by determining absorbance at 690 nm. The cell density and biomass (dry weight) were obtained by using pre-determined correlation between optical density and cell density, and between cell density and dry weight.

As summarized in Table 1, below, for the first run, the initial feed volume of microalgae was 857 mL with a cell density of 331.9 mg/L. After 30 minutes of continuous operation at a feed flow rate of 704 mL/min and a differential pressure across the membrane of 15 in Hg, the feed volume was reduced to a volume of 184 mL. Thus, the concentrating process was stopped.

To recover the concentrated microalgae from the membrane, the feed was circulated through the membrane at an elevated flow rate and without vacuum applied at permeate port. This breaks down the “algae cake” deposited along the membrane and increases the recovery rate. Thus, the peristaltic pump speed was increased to 1020 mL/min from the operating speed of 704 mL/min, and the feed circulation was continued for 5 minutes. Then, the pump was run in reverse to gather the concentrated cell suspension to the feed container. The concentrated cell suspension was measured to be 184 mL with a cell density of 1,589.1 mg/L, which is 5 times higher than the original suspension. Eighty percent of the culture medium was recovered by the process.

After removing the concentrated cells, the membrane was regenerated by flushing it three times with 200 mL of recovered culture medium. This process was found to be sufficient to bring the water flux back to the initial baseline. This process also recovered the residual biological material trapped in the system (less than 8%), which can be used as seed for next batch culture.

The viability of the recovered residual microalgae and concentrated microalgae were verified and both exhibited normal growth rate. Therefore, the harvesting method disclosed herein did no harm to the microalgae cells.

Then, a second and third run for cyanobacteria cell harvesting were performed using the same procedure and feed volume, but different biomass densities and feed flow rates. The second and third runs were performed at a flow rate of 1020 mL/min. The feed descriptions and results are also set forth in Table 1 below.

TABLE 1

| | Description | Vol (ml) | Density (M. cells/ml) | Concentration (mg/L) | Amount (M. cells) |
|---|---|---|---|---|---|
| 1st Run | Starting Cells | 857 | 33.19 | 331.9 | 28443.6 |
| | Final concentrated cells | 175 | 158.91 | 1589.1 | 27809.7 |
| | Combined permeates | 665 | ND | ND | ND |
| | Cells trapped in the system | 190 | 7.20 | 72.0 | 1368.0 |
| | Filtration efficiency | | | | ~100% |
| | Recovery rate | | | | 97.8% |
| | Concentration fold | | | | 4.8 |
| 2nd Run | Starting cells | 860 | 13.66 | 136.6 | 11746.2 |
| | Final concentrated cells | 170 | 67.55 | 675.5 | 11483.5 |
| | Combined permeates | 670 | ND | ND | ND |
| | Cells trapped in the system | 190 | 6.4 | 64 | 1216.0 |
| | Filtration efficiency | | | | ~100% |
| | Recovery rate | | | | 97.8% |
| | Concentration fold | | | | 4.9 |
| 3rd Run | Starting cells | 860 | 19.25 | 192.5 | 16555.0 |
| | Final concentrated cells | 230 | 66.89 | 668.9 | 15384.7 |
| | Combined permeates | 619 | ND | ND | ND |
| | Cells trapped in the system | 190 | 5.73 | 57.3 | 1088.7 |
| | Filtration efficiency | | | | ~100% |
| | Recovery rate | | | | 92.9% |
| | Concentration fold | | | | 3.5 |

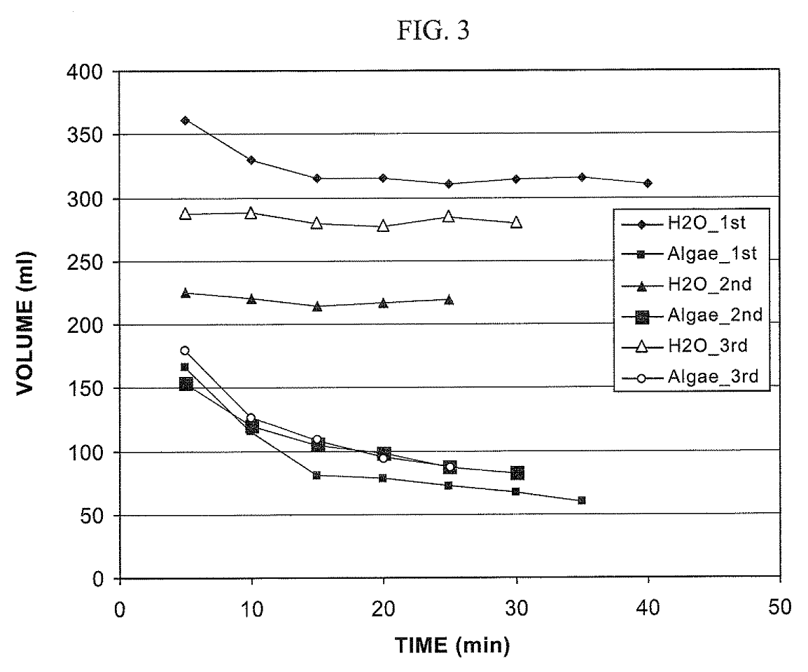

The flux of distilled water was measured before and after each run of microalgae harvesting and used to evaluate the membrane performance. FIG. 3 shows a comparison of flux for water and cyanobacteria suspensions from the three separate runs. The results indicate that the performance of the ceramic membrane filter was consistent in three separate runs. The water fluxes measured after three runs remained in the normal range (220 mL to 350 mL). The filtration efficiency, calculated by comparing the cell density in permeate and in the final concentrate [concentrate/(concentrate +filtrate)], was nearly 100%, as the cells in the combined filtrates/permeates were undetectable.

The membrane filter of Example 1 is capable of concentrating 50 g/m² continuously, without significant change in filtration flux (100 L/hr.m².bar determined at a velocity of 238.1 cm/min). When the water flux dropped to 30% of the baseline, the filter was removed and soaked in commercial bleach for 30 minutes, and then rinsed with distilled water three times. This treatment was able to restore the membrane performance and no significant membrane fouling was observed. With such practice, cumulatively, a total of 8 grams of microalgae biomass (dry weight) was harvested by the 1″×2″ membrane filter, which is equivalent to 360 g/m².

### COMPARATIVE EXAMPLE

Two comparative runs of cell harvesting using a traditional method, centrifugation at 8,000 rpm for 5 minutes, were also performed. The cell suspensions used for the comparative runs were the same as that identified as “2nd Run” in Table 1. Recovery rate was calculated based on the cell numbers detected in pellet and supernatant. The results of the two comparative centrifugation runs are set forth in Table 2.

TABLE 2

| | OD690 | Biomass (million cells per ml) | volume (ml) | Biomass (million cells) | Recovery/concentration rate |
|---|---|---|---|---|---|
| Start | 0.771 | 13.74216 | 40 | 549.69 | |
| Sup #1 | 0.125 | 2.91973 | 39 | 113.87 | 79.3% |
| Supernatant #2 | 0.155 | 3.42232 | 39 | 133.47 | 75.7% |

The recovery rate for methods of the present invention, i.e., (biomass in concentrate+residual biomass)/starting total biomass, set forth in Table 1, are consistently higher than 90%; whereas, the comparative runs, i.e., the traditional centrifugation methods, gave recovery rates of less than 80%, calculated as biomass in precipitate/starting total biomass.

### Example 2

Synechocystis ps. PCC 6803, a unicellular blue-green algae, was harvested from a cell suspension cultured in the modified BG-11 medium (ATCC medium 616) plus 10 mM Hepes buffer (pH 7.4) to maintain the pH, using membrane filter SP-2-1, as described in Example 1.

A series of cell suspensions of varying density were filtered, the lowest sample cell concentration being 100 mg/L and the highest being 1,800 mg/L, using the experimental set up described above in Example 1. The results are set forth in Table 3 below. Synechocystis ps. PCC 6803 biomass was concentrated to as high as 5349 mg /L using a 1″×2″ membrane filter.

TABLE 3

| | Start density | End Density | Algae flux | Velocity |
|---|---|---|---|---|
| Run | (mg/L) | (mg/L) | (L/hr · m2 · bar) | (cm/min) |
| 1 | 104.3 | 652.8 | 105.6 | 238.1 |
| 2 | 121.7 | 486.9 | 79.3 | 238.1 |
| 3 | 241.4 | 848.2 | 81.4 | 238.1 |
| 4 | 250.1 | 799.6 | 59.7 | 238.1 |
| 5 | 146.5 | 768.4 | 110.7 | 388.0 |
| 6 | 331.9 | 1589.1 | 127.8 | 388.0 |
| 7 | 216.2 | 963.8 | 80.4 | 463.0 |
| 8 | 267.5 | 1173.9 | 99.7 | 463.0 |
| 9 | 376.1 | 1421.6 | 70.7 | 463.0 |
| 10 | 444.4 | 1546.6 | 81.6 | 463.0 |
| 11 | 659.5 | 2233.5 | 50.5 | 463.0 |
| 12 | 1052.6 | 2153.9 | 64.1 | 463.0 |
| 13 | 1069.2 | 5114.4 | 55.5 | 463.0 |
| 14 | 1772.8 | 5348.9 | 41.9 | 463.0 |
| 15 | 136.6 | 742.6 | 148.5 | 582.0 |
| 16 | 192.5 | 668.9 | 167.6 | 582.0 |
| 17 | 575.7 | 1055.9 | 81.9 | 628.3 |

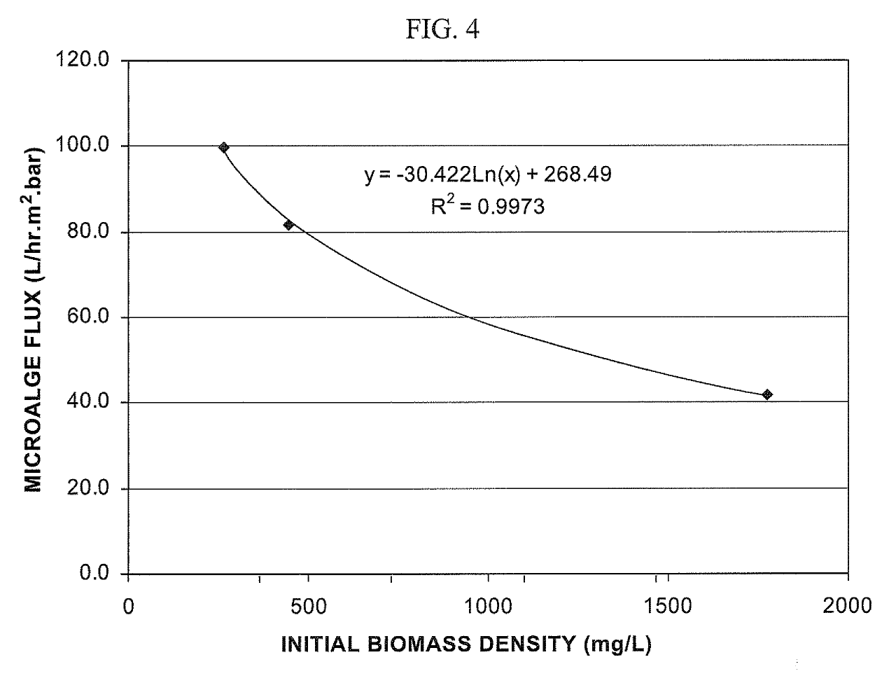

Using the data from the samples set forth in Table 3 with a constant velocity of 463 cm/min, the impact of biomass density on filtration flux is shown in FIG. 4. As seen in FIG. 4, in general, the filtration flux (L/hr.m².bar) decreases as initial biomass density (mg/L) increases. For example, when initial biomass density increased 7 fold, the filtration flux decreased by 58%.

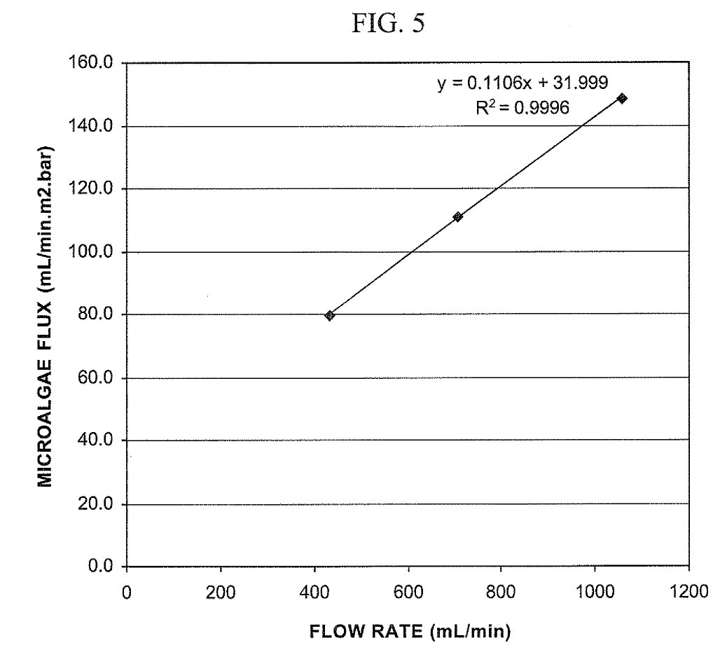

The filtration flux was also affected by feed flow rate. As shown in FIG. 5, using the data from samples in Table 3 having initial biomass densities of 130 mg/L, the increase of filtration flux is proportional to the increase of the feed flow rate through the membrane module. For example, as the feed flow rate was increased 2.4 fold, the filtration flux increased 1.9 fold.

### Example 3

Five ceramic membrane filters (1″×2″ module) were tested under the same operating conditions to study higher filtration flux: SP-2-1, SP-2-2, SP-2-3, SP-2-4, and SP-2-8. The filters have similar dimensions and membrane mean pore size (0.2˜0.4 μm) as described in Example 1 above; however, they vary in porosity and mean pore size of the ceramic support. The filtration surface area for these membrane filters is 210.5 cm² and the area of cross section or front area is 1.8144 cm². Synechocystis ps PCC 6803 cells at a density of 350 mg/L were used for testing. Vacuum of 0.5 bar was applied at the permeate port and feed velocity was maintained at 401.7 cm/min. Table 4 summarizes the porosity and mean pore size of these membrane filters and their respective filtration efficiency and flux.

TABLE 4

| | porosity of | mean pore size of ceramic | filtration | water flux | Algae Flux |
|---|---|---|---|---|---|
| Membrane Filters | membrane (%) | support (μm) | efficiency | (mL/min · m² · bar) | (mL/min · m² · bar) |
| SP-2-1 | 49.6 | 9.0 | 97.7% | 5955.6 | 1451.1 |
| SP-2-2 | 43.8 | 9.4 | 96.7% | 5222.2 | 1203.7 |
| SP-2-3 | 41.3 | 10.7 | 94.9% | 6111.1 | 1944.4 |
| SP-2-4 | 37.8 | 9.2 | 96.6% | 2111.1 | 1348.1 |
| SP-2-8 | 37.3 | 17.7 | 95.6% | 1333.3 | 381.5 |

The results set forth in Table 4 suggest that the selection of the porosity and pore size distribution of the monolithic body may be important for achieving high flux as well as high filtration efficiency. For example, high flux was detected when the total porosity was within the range of 38 to 50% and the mean pore size of the ceramic support was within the range of 7 to 10 μm. Large pore size, on the other hand, may result in reduced flux due to the thicker membrane coating that is necessary to cover larger pores. For example, as shown in Table 4, the filter SP-2-8, with the large monolith pore size of 17.7 μm, exhibited very low flux.

### Example 4

Spirulina platensis is a planktonic photosynthetic filamentous cyanobacterium. Due to its highly nutritional nature (i.e., containing high protein and lipids), this species has been used for food and nutritional supplements. Even though it is single-celled, Spirulina is relatively large, attaining sizes of 0.5 mm in length, which is about 100 times the size of most other algae. Spirulina is cultured in alkaline condition (pH 9 to 11). Filtration of this species was performed to study the performance of a membrane filter with large size microalgae and the tolerability of the filter to high pH.

The membrane filter used for this experiment was SP-2-1, as described in Example 1. The feed Spirulina cell density was 188 mg/L. After filtration for 25 min at a feed flow rate of 840 mL/min and a differential pressure across the membrane of 15 in Hg, the feed volume was reduced to 188 mL from the initial 600 mL, and the biomass density was increased to 686 mg/L. The resulting filtration flux was 98.9 L/hr.m².bar. This data is similar to the filtration flux in Example 2, with the small size cell species, Synechocystis ps. PCC 6803, compared under similar filtration conditions. Therefore, it can be seen that, for the same membrane filter, the filtration flux may not, in at least some embodiments, be affected by the size or shape of the biological material, for example ranging from 3 μm in diameter to greater than 500 μm in length.

### Example 5

The effect of a driving force applied across the membrane was studied in this example. A larger mullite-based membrane filter was used: 12 inches in length, 1 inch in diameter, and with 85 round fluid passageways (1.7 mm in diameter). The total filtration surface area was 0.13 m², and the open frontal area of was 1.88 cm². The ceramic support had a total porosity of 37.7%, with a 4.2 μm mean pore size. The membrane coated on top of the support, which was alumina, had a 0.2 μm mean pore size.

Due to the high filtration flux of the membrane filter, no vacuum pressure was applied at the permeate port. The measured differential pressure across the membrane was 0.057 bar. The microalgae suspension, Synechocystis ps. PCC 6803, was circulated at a high feed flow rate, 1595 mL/min, for 30 min. The biomass density was concentrated from 329.4 mg/L to 1379.1 mg/L; while the feed volume was reduced from 800 mL to 191 mL. The achieved filtration efficiency was 97% or greater.

In another run, the filtration was run at low flow rate of 738 mL/min, and a vacuum of 10 in Hg was applied to the permeate port. At a feed density of 325 mg/L, the filtration flux was 5,271 mL/min.m².bar

What is claimed is:

1. A method for harvesting microalgae, said method comprising passing at least one biological suspension comprising at least one biological material comprising microalgae through at least one membrane filter;
wherein the at least one membrane filter is comprised of a monolithic body comprising porous material.

2. The method of claim 1, wherein the at least one biological suspension is further comprised of at least one liquid or slurry chosen from culture medium obtained from open ponds and/or from enclosed photobioreactors, oils, and organic solvents.

3. The method of claim 1, wherein the fluid volume of the at least one biological suspension is reduced by 80 percent or greater.

4. The method of claim 1, wherein the at least one biological suspension has a pH ranging from 2 to 13.

5. The method of claim 1, wherein the size of the at least one biological material is greater than 0.2 μm.

6. The method of claim 1, wherein the biomass density of the at least one biological suspension ranges from 10 mg/L to 5000 mg/L prior to harvesting.

7. The method of claim 1 which further comprises applying at least one driving force for passing the at least one biological suspension through the at least one membrane filter, wherein said driving force ranges from 0.05 bar to 4 bar.

8. The method of claim 1, further comprising at least one step of recovering concentrated and/or residual biological material.

9. The method of claim 8, wherein the at least one step of recovering concentrated and/or residual biological material further comprises circulating the harvested biological material through the system at an increased feed flow rate.

10. The method of claim 8, wherein the at least one step of recovering concentrated and/or residual biological material further comprises reversing the flow through the at least one membrane filter.

11. The method of claim 8, wherein the residual biological material is flushed from the membrane filter using recycled or fresh culture medium.

12. The method of claim 11, further comprising using viable recovered residual biological material to start a new cycle of biomass production.

13. A method for harvesting biological material, said method comprising passing at least one biological suspension comprising at least one biological material through at least one membrane filter;
wherein the at least one membrane filter is comprised of a monolithic body comprising ceramic material; and
wherein the monolithic body comprises at least one filtrate conduit.

14. The method of claim 13, wherein the at least one biological suspension is passed through the at least one membrane filter two or more times.

15. The method of claim 13, wherein said ceramic material is comprised of mullite.

16. The method of claim 15, wherein the ceramic material has a porosity ranging from 37% to 50% and pore size distribution ranging from 4 μm to 10 μm.

17. The method of claim 13, wherein the ceramic material comprises fluid passageways coated with at least one membrane layer, wherein the membrane layer is comprised of porous material having smaller pore sizes than the pores of the monolithic body.

18. The method of claim 17, wherein the membrane layer is comprised of porous material having pore sizes ranging from 0.2 to 0.4 μm.

19. The method of claim 13, further comprising at least one step of cleaning and/or regenerating the at least one membrane filter chosen from thermal treatment, chemical treatment, and sterilization.

20. The method of claim 13, wherein the filtration efficiency of the method is greater than 80 percent.

21. The method of claim 13, wherein the recovery rate of the method is greater than 80 percent.

22. A method for harvesting microalgae for an application chosen from biofuel, food, and nutritional supplements, said method comprising:
passing at least one biological suspension comprising at least one biological material comprising microalgae through at least one membrane filter; and
further treating the concentrated at least one biological material to make a product chosen from biofuel, food, and nutritional supplements;
wherein the at least one membrane filter is comprised of a monolithic body comprising ceramic material.
